(19) United States
(12) Patent Application Publication
(10) Pub. No.: US 2011/0201587 A1
(43) Pub. Date: Aug. 18, 2011

(54) HSP90 INHIBITORS AND METHODS OF USE

(75) Inventor: Leland Shapiro, Denver, CO (US)

(73) Assignee: BIO HOLDING, INC., Wilson, NC (US)

(21) Appl. No.: 12/706,613

(22) Filed: Feb. 16, 2010

(51) Int. Cl. A61K 31/395; A61P 31/04; A61P 31/12; A61P 31/10; A61P 19/02; A61P 9/00; A61P 25/00; A61P 1/00; A61P 1/18; A61P 11/00; A61P 13/12; A61P 37/06

(52) U.S. Cl. 514/183

(57) ABSTRACT

The present invention provides methods for treating various clinical conditions associated with biological activity of HSP90 in a subject. Such methods include administering to the subject in need of such a treatment a therapeutically effective amount of a composition comprising an HSP90 inhibitor.

### FIELD OF THE INVENTION

The present invention relates to methods for treating various clinical conditions using an HSP90 inhibitor.

### BACKGROUND OF THE INVENTION

Heat shock proteins, as a class, are among the most highly expressed cellular proteins across all species. As their name implies, heat shock proteins protect cells when stressed by elevated temperatures. They account for 1-2% of total protein in unstressed cells. However when cells are heated, the fraction of heat shock proteins increases to 4-6% of cellular proteins.

Heat shock protein 90 (Hsp90) is one of the most common of the heat related proteins. The protein is named “HSP” for obvious reasons whereas the “90” comes from the fact that it weighs roughly 90 kiloDaltons. A 90 kDa size protein is considered a fairly large for a non-fibrous protein.

The function of Hsp90 includes assisting in protein folding, cell signaling, and tumor repression. This protein was first isolated by extracting proteins from stressed cells. These cells were stressed by heating, dehydrating or by other means, all of which caused the cell's proteins to begin to denature.

Hsp90 is highly conserved and expressed in a variety of different organisms from bacteria to mammals—including the prokaryotic analogue htpG (high temperature protein G) with 40% sequence identity and 55% similarity to the human protein. Yeast Hsp90 is 60% identical to human Hsp90α. In mammalian cells, there are two or more genes encoding cytosolic Hsp90 homologues, with the human Hsp90α showing 85% sequence identity to Hsp90β.

In unstressed cells, Hsp90 plays a number of important roles, which include assisting in folding, intracellular transport, maintenance, and degradation of proteins as well as facilitating cell signaling. Hsp90 is also known to associate with the non-native structures of many proteins which has lead to the theory that Hsp90 is involved in protein folding in general. Furthermore Hsp90 has been shown to suppress the aggregation of a wide range of “client” or “substrate” proteins and hence acts as a general protective chaperone.

While most therapeutic agents of HSP90 has been focused on treatment of cancer, the present inventor have discovered HSP90 is responsible for various other diseases. Accordingly, the present invention is directed to treating a variety of clinical conditions which are mediated by HSP90.

### SUMMARY OF THE INVENTION

The present invention is based on the discovery by the present inventor that besides tumor genesis, the presence of HSP90 in a subject is also responsible for previously unrecognized clinical conditions. Accordingly, some aspects of the invention provide methods for treating various clinical conditions associated with HSP90. Such methods of the invention typically comprise inhibiting HSP90 by administering to the subject in need of such a treatment a therapeutic amount of a composition comprising an HSP90 inhibitor. The clinical condition is typically selected from the group consisting of systemic disease; bacterial infection; fungal infection; a disease or disorder of a joint, cardiovascular system, central nervous system, lung, pancreas, kidney, gastrointestinal tract, or a limb; autoimmune disease; hematologic disease and a combination thereof.

In some embodiments, the bacterial infection comprises infection of mycobacteria, anthrax, bacterial pneumonia, or a combination thereof.

Yet in other embodiments, the fungal infection comprises infection of candida or aspergillus, mucor, fusarium, or a combination thereof.

Still in other embodiments, the disease or disorder of a joint comprises rheumatoid arthritis, degenerative joint disease (e.g., osteoarthritis), gout, seroneagative spondyloarthropathies, or a combination thereof.

In other embodiments, the disease or disorder of central nervous system comprises stroke, transient ischemic attack, intracerebral hemorrhage, Parkinson disease, amyotropic lateral sclerosis, multiple sclerosis, viral or bacterial meningitis or encephalitis, or a combination thereof.

Yet in other embodiments, the disease or disorder of lung comprises acute respiratory distress syndrome, shock lung, asthma, chronic obstructive pulmonary disease, primary pulmonary hypertension, pulmonary embolization, or a combination thereof.

Still in other embodiments, the disease or disorder of liver comprises shock liver, drug-induced hepatitis, viral hepatitis, or a combination thereof.

Yet still in other embodiments, the disease or disorder of pancreas comprises acute pancreatitis, diabetes mellitus type II, chronic pancreatitis, or a combination thereof.

In other embodiments, the disease or disorder of kidney comprises pre-renal azotomia, acute tubular necrosis, ischemic nephropaty, glomerulonephritis, interstitial nephritis, end-stage renal disease, or a combination thereof.

In some embodiments, the disease or disorder of gastrointestinal tract comprises ischemic bowel, bowel infarction, inflammatory bowel disease, celiac disease, or a combination thereof.

Still in other embodiments, the disease or disorder of the limb comprises limb ischemia, limb infarction, thromboangiitis obliterans (Buerger's disease), Raynaud phenomenon, Raynaud disease, peripheral ulcer disease, or a combination thereof.

Yet in other embodiments, the systemic disease comprises sarcoidosis, vasculitis, systemic lupus erythematosis, crest syndrome, progressive systemic sclerosis, Sjogren's syndrome, disease, ankylosing spondylitis, or a combination thereof.

Exemplary HSP90 inhibitors that are useful include, but are not limited to, retaspimycin, tanespimycin, geldanamycin derivative, SNX-2112, SNX-5422, STA-9090, AUY922, or a derivative or a pro-drug thereof, or a combination thereof.

Other aspects of the invention include methods for treating a clinical condition associated with excessive nitric oxide. The present inventor has discovered that excessive HSP90 leads to excessive nitric oxide production. Methods of these aspects of the invention include inhibiting HSP90 in a subject in need of such a treatment by administering a therapeutically effective amount of a composition comprising an HSP90 inhibitor. Typically, subjects that are in need of such a treatment are those whose clinical condition(s) are manifested due to excessive nitric oxide production.

In some embodiments of such aspects of the invention, the clinical condition associated with excessive nitric oxide comprises acquired tubulointerstitial disease, acute pancreatitis, acute respiratory failure, acute respiratory distress syndrome (ARDS), age-associated memory impairment, airway inflammation, amyotrophic lateral sclerosis, asthma, atherosclerosis, autoimmune disease, myocarditis, cerebral ischemia, cerebrovascular disease, chronic liver disease, chronic lung disease, chronic obstructive pulmonary disease, chronic otitis media, congestive heart failure, coronary artery disease, coronary artery ectasia, dysfunctional uterine bleeding, dysmenorrhea, endotoxic shock, end-stage renal disease, falciparum malaria, gastrointestinal pathophysiology, glaucoma, glutamate-induced asthma, glutamate induced Chinese restaurant syndrome, heart failure, heat stress, gastritis, Hirschsprung's disease, hypertension, hypoxemic respiratory failure, inflammatory arthritis, inflammatory bowel disease, inflammatory joint diseases, liver cirrhosis, liver disease, Lyme neuroborreliosis, migraine, neonatal and pediatric respiratory failure, nephrotoxicity, neurodegenerative diseases, orthopedic disease, osteoarthritis, oxidant stress, Parkinson's disease, pediatric pulmonary disease, pleural inflammation, preeclampsia, primary ciliary dyskinesia, primary pulmonary hypertension, protozoan infections, pulmonary hypertension, retinal disease, septic shock, sickle cell anemia, stroke (hemorrhagic or non-hemorrhagic), systemic lupus erythematosus, traumatic brain injury, or a vascular disease.

Still in other embodiments, the HSP90 inhibitor comprises retaspimycin, tanespimycin, geldanamycin derivative, SNX-2112, SNX-5422, STA-9090, AUY922, or a derivative or a pro-drug thereof, or a combination thereof.

Yet other aspects of the invention provide methods for treating a clinical condition associated with HSP90 in a subject. In these aspects of the invention, the clinical condition associated with HSP90 is selected from the group consisting of anthrax toxicity, bacterial infection, ischemia-reperfusion injury, and a combination thereof. Methods of these aspects of the invention include administering a therapeutically effective amount of a composition comprising an HSP90 inhibitor to a subject in need of such a treatment.

In some embodiments, the HSP90 inhibitor comprises retaspimycin, tanespimycin, geldanamycin derivative, SNX-2112, SNX-5422, STA-9090, AUY922, or a derivative or a pro-drug thereof, or a combination thereof.

### BRIEF DESCRIPTION OF THE DRAWINGS

FIG. 1A is a graph showing the results of inhibition of LPS- or IL-18-induced HIV production using an HSP90 inhibitor at various concentrations.

FIG. 1B is a graph showing the results of inhibition of HIV infection of peripheral blood mononuclear cells (PBMC) by an HSP90 inhibitor at various concentrations. PBMC from five healthy patients were infected for 4 hr with either a T- or M-tropic strain of HIV. Following infection, the PBMC were incubated for 3 days in the presence or absence of geldanamycin and HIV p24 antigen was measured as an indicator of HIV replication.

### DETAILED DESCRIPTION OF THE INVENTION

### Definitions

The terms “pro-drug” and “prodrug” are used interchangeably herein and refer to any compound which releases an active HSP90 inhibitor in vivo when such prodrug is administered to a mammalian subject. HSP90 inhibitor prodrugs are typically prepared by modifying one or more functional group(s) present in the HSP90 inhibitor in such a way that the modification(s) can be cleaved in vivo to release the parent compound. Prodrugs of HSP90 inhibitor include compounds where a hydroxy, amino, or sulfhydryl group in a HSP90 inhibitor is bonded to any group that can be cleaved in vivo to regenerate the free hydroxyl, amino, or sulfhydryl group, respectively. Examples of prodrugs include, but are not limited to, esters (e.g., acetate, formate, and benzoate derivatives), carbamates (e.g., N,N-dimethylaminocarbonyl) of hydroxy functional groups in a HSP90 inhibitor, and the like.

“A therapeutically effective amount” means the amount of a substance that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease (prophylaxis), i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

“Clinical condition associated with HSP90” means that the clinical condition manifested by a subject is at least in part caused by HSP90 and that can be benefited by HSP90 blockade.

### Heat Shock Proteins (HSPs)

Molecular chaperones maintain the appropriate folding and conformation of proteins and are important in regulating the balance between protein synthesis and degradation. They have been shown to be important in regulating many important cellular functions, such as cell proliferation and apoptosis.

Exposure of cells to a number of environmental stresses, including heat shock, alcohols, heavy metals and oxidative stress, results in the cellular accumulation of a number of chaperones, commonly known as heat shock proteins (HSPs). Induction of HSPs protects the cell against the initial stress insult, enhances recovery and leads to maintenance of a stress tolerant state. It has also become clear, however, that certain HSPs may also play a major molecular chaperone role under normal, stress-free conditions by regulating the correct folding, degradation, localization and function of a growing list of important cellular proteins.

A number of multigene families of HSPs exist, with individual gene products varying in cellular expression, function and localization. They are classified according to molecular weight, e.g., HSP70, HSP90, and HSP27.

Several diseases in humans can be acquired as a result of chaperone-associated protein misfolding or excessive function of a normal protein. In some conditions, misfolded proteins can cause protein aggregation resulting in neurodegenerative disorders. Also, misfolded proteins may result in loss of wild type protein function, leading to deregulated molecular and physiological functions in the cell.

HSPs have also been implicated in cancer. For example, there is evidence of differential expression of HSPs which may relate to the stage of tumor progression. As a result of the involvement of HSP90 in various critical oncogenic pathways and the discovery that certain natural products with anticancer activity are targeting this molecular chaperone, many of ordinary skilled in the art have been developing methods to inhibit HSP function to treat cancer.

### HSP90

It is believed that HSP90 constitutes about 1-2% of total cellular protein, and is usually present in the cell as a dimer in association with one of a number of other proteins. It is essential for cell viability, and it exhibits dual chaperone functions. It plays a key role in the cellular stress response by interacting with many proteins after their native conformation has been altered by various environmental stresses, such as heat shock, ensuring adequate protein folding and preventing non-specific aggregation. In addition, recent results suggest that HSP90 may also play a role in buffering against the effects of mutation, presumably by correcting the inappropriate folding of mutant proteins. However, HSP90 also has an important regulatory role. Under normal physiological conditions, together with its endoplasmic reticulum homologue GRP94, HSP90 plays a housekeeping role in the cell, maintaining the conformational stability and maturation of several key client proteins. These can be subdivided into three groups: (a) steroid hormone receptors, (b) Ser/Thr or tyrosine kinases (e.g., ERBB2, RAF-1, CDK4, and LCK), and (c) a collection of apparently unrelated proteins, e.g., mutant p53 and the catalytic subunit of telomerase hTERT. All of these proteins play key regulatory roles in many physiological and biochemical processes in the cell.

The highly conserved HSP90 family in humans consists of four genes, namely the cytosolic HSP90α and HSP90β isoforms, GRP94 in the endoplasmic reticulum and HSP75/TRAP1 in the mitochondrial matrix. It is thought that all the family members have a similar mode of action, but bind to different client proteins depending on their localization within the cell. For example, ERBB2 is known to be a specific client protein of GRP94 and type 1 tumour necrosis factor receptor (TNFR1) and RB have both been shown to be clients of TRAP1.

HSP90 participates in a series of complex interactions with a range of client and regulatory proteins. Although the precise molecular details remain to be elucidated, biochemical and X-ray crystallographic studies carried out over the last few years have provided increasingly detailed insights into the chaperone function of HSP90.

It is believed that HSP90 is an ATP-dependent molecular chaperone, with dimerization of the nucleotide binding domains being essential for ATP hydrolysis, which is in turn essential for chaperone function. Binding of ATP results in the formation of a toroidal dimer structure in which the N terminal domains are brought into closer contact with each other resulting in a conformational switch known as the “clamp mechanism”.

### HSP90 Inhibitors

The first class of HSP90 inhibitors to be discovered was the benzoquinone ansamycin class, which includes the compounds herbimycin A and geldanamycin. They were shown to reverse the malignant phenotype of fibroblasts transformed by the v-Src oncogene, and subsequently to exhibit potent antitumour activity in both in vitro and in vivo animal models.

Immunoprecipitation and affinity matrix studies have shown that the major mechanism of action of geldanamycin involves binding to HSP90. Moreover, X-ray crystallographic studies have shown that geldanamycin competes at the ATP binding site and inhibits the intrinsic ATPase activity of HSP90. This in turn prevents the formation of mature multimeric HSP90 complexes capable of chaperoning client proteins. As a result, the client proteins are targeted for degradation via the ubiquitin proteasome pathway. 17-Allylamino, 17-demethoxygeldanamycin (17AAG) retains the property of HSP90 inhibition resulting in client protein depletion and antitumour activity in cell culture and xenograft models, but has significantly less hepatotoxicity than geldanamycin. 17AAG is currently being evaluated in Phase I clinical trials.

Radicicol is a macrocyclic antibiotic shown to reverse the malignant phenotype of v-Src and v-Ha-Ras transformed fibroblasts. It was shown to degrade a number of signalling proteins as a consequence of HSP90 inhibition. X-ray crystallographic data confirmed that radicicol also binds to the N terminal domain of HSP90 and inhibits the intrinsic ATPase activity. Radicicol lacks antitumour activity in vivo due to the unstable chemical nature of the compound.

Coumarin antibiotics are known to bind to bacterial DNA gyrase at an ATP binding site homologous to that of the HSP90. The coumarin, novobiocin, was shown to bind to the carboxy terminus of HSP90, i.e., at a different site to that occupied by the benzoquinone ansamycins and radicicol which bind at the N-terminus. However, this still resulted in inhibition of HSP90 function and degradation of a number of HSP90-chaperoned signalling proteins. Geldanamcyin cannot bind HSP90 subsequent to novobiocin; this suggests that some interaction between the N and C terminal domains must exist and is consistent with the view that both sites are important for HSP90 chaperone properties.

A purine-based HSP90 inhibitor, PU3, has been shown to result in the degradation of signalling molecules, including erb-B2, and to cause cell cycle arrest and differentiation in breast cancer cells.

Other HSP90 inhibitors are also known. See for example, U.S. Pat. No. 7,612,201.

### HSP90 as a Therapeutic Target

Due to its involvement in regulating a number of signaling pathways that are crucially important in driving the phenotype of a tumor, and the discovery that certain bioactive natural products exert their effects via HSP90 activity, the molecular chaperone HSP90 is currently being assessed as a new target for anticancer drug development.

The predominant mechanism of action of geldanamycin, 17AAG, and radicicol involves binding to HSP90 at the ATP binding site located in the N-terminal domain of the protein, leading to inhibition of the intrinsic ATPase activity of HSP90.

Inhibition of HSP90 ATPase activity prevents recruitment of co-chaperones and encourages the formation of a type of HSP90 heterocomplex from which these client proteins are targeted for degradation via the ubiquitin proteasome pathway.

Treatment with HSP90 inhibitors leads to selective degradation of important proteins involved in cell proliferation, cell cycle regulation and apoptosis, processes which are fundamentally important in cancer.

Inhibition of HSP90 function has been shown to cause selective degradation of important signalling proteins involved in cell proliferation, cell cycle regulation and apoptosis, processes which are fundamentally important and which are commonly deregulated in cancer. An attractive rationale for developing drugs against this target for use in the clinic is that by simultaneously depleting proteins associated with the transformed phenotype, one may obtain a strong antitumor effect and achieve a therapeutic advantage against cancer versus normal cells. These events downstream of HSP90 inhibition are believed to be responsible for the antitumor activity of HSP90 inhibitors in cell culture and animal models.

Others have indicated using HSP90 inhibitors in the treatment of diseases such as cancers; viral diseases such as Hepatitis C (HCV); Immunosupression such as in transplantation; Anti-inflammatory diseases such as Rheumatoid arthritis, Asthma, MS, Type II Diabetes, Lupus, Psoriasis and Inflammatory Bowel Disease; Cystic fibrosis; Angiogenesis-related diseases, diabetic retinopathy, haemangiomas, psoriasis, endometriosis and tumor angiogenesis.

### METHODS OF THE INVENTION

The present inventor have discovered that HSP90 is also responsible for nitric oxide (NO) production. Accordingly, some aspects of the invention provide methods for treating a clinical condition associated with excessive nitric oxide. Nitric oxide (NO), also known as endothelium-derived relaxing factor (EDRF), is a potent vasodilator, oxidant, and neurotransmitter produced by many different types of cells and tissues, such as endothelium, macrophages and neuronal cells.

It is believed that the NO synthase enzymes (NOS) exist in at least three isoforms, namely, neuronal constitutive NOS(N-cNOS) which is present constitutively in neurons, endothelial constitutive NOS (E-cNOS) which is present constitutively in endothelial cells, and inducible NOS (iNOS) which is expressed following stimulation by cytokines and lipopolysaccharides in macrophages and many other cells. Among these three isoforms, N-cNOS and E-cNOS are calcium-dependent whereas iNOS is calcium-independent. NO synthesized by nitric oxide synthase from arginine and oxygen is also an important signal transducing molecule in various cell types. In macrophages NO has assumed, under certain situations, the role of a cytotoxic agent—a reactive nitrogen intermediate that is lethal to cancer cells and microorganisms. The release of nitric oxide is also involved in other acute and chronic inflammatory diseases. These diseases include but are not limited to diseases such as, for example, acute and chronic infections (viral, bacterial and fungal), acute and chronic bronchitis, sinusitis, and upper respiratory infections, including the common cold; acute and chronic gastroenteritis and colitis; acute and chronic cystitis, and urethritis; acute and chronic dermatitis; acute and chronic conjunctivitis; acute and chronic serositis (pericarditis, peritonitis, synovitis, pleuritis and tendinitis); uremic pericarditis; acute and chronic cholecystitis; acute and chronic vaginitis; drug reactions; insect bites; burns and sunburn.

Released NO combines very rapidly with superoxide to form peroxynitrite (ONOO^{-•}), a reactive tissue damaging nitrogen species thought to be involved in the pathology of several chronic diseases. Nitric oxide also inhibits iron-containing enzymes important in respiration and DNA synthesis. Peroxynitrite decomposes to the reactive NO₂ and hydroxyl radicals, and NO stimulates ADP-ribosylation of various proteins including glyceraldehyde-3-phosphate dehydrogenase, with consequent inactivation.

Many proteins are reported to modulate NO production. Macrophage deactivating factor and TGF-β partially blocked NO release by macrophages activated with γ-interferon (γ-IFN or IFN-γ) and TGF-α (transforming growth factor-α), but not when activated by γ-IFN and lipopolysaccharide (LPS or endotoxin). Epidermal growth factor can suppress both NO and H₂O₂ production by keratinocytes. Incubation of LPS-activated peritoneal neutrophils with IL-8 blocks both the release of NO and NOS induction at the transcriptional level.

TGF-β₁ and 12-O-tetradecanoylphorbol-13-acetate (i.e., phorbol myristyl acetate or PMA) inhibit LPS and γ-IFN-induced NO synthesis in mouse bone marrow cells. In contrast, in bovine pigmented retinal epithelial cells TGF-β increases the NO production, as measured by nitrite, attributable to treatment with LPS and 7-IFN. In this system both fibroblast growth factor (FGF)-1 and FGF-2 inhibit nitrite production, likely by inhibiting the induction of NOS mRNA at the transcriptional level. Insulin-like growth factor 1 reduces the amount of NO produced by the action of IL-1β on vascular smooth muscle cells. The fact that so many agents can modulate NO activity by increasing or inhibiting NO production suggests that NO production may be important in many different contexts.

The overproduction in the body of nitric oxide (NO) and/or peroxynitrite has been suggested by some to be a contributing factor to diseases that are immune-mediated and/or inflammatory. In a clinical study the levels of IL-6, IL-1β, and NO were shown to be involved in the pathogenesis of scorpion envenomation and correlated with the severity of envenomation. An extensively used model system to study multiple sclerosis, an example of a disease treated by the present invention, is experimental allergic encephalomyelitis (EAE) in rats and mice.

None of the prior art recognized that HSP90 inhibitors might in fact prevent NO synthesis. It is believed that therapeutic and physiological levels of an HSP90 inhibitor efficiently blocks γ-IFN- and LPS-induced NO synthesis. Thus some aspects of the invention provide methods for safe and effective amelioration of many diseases related to a clinical condition associated with excessive nitric oxide.

Exemplary clinical conditions for which the present inventor have found to be treatable by an HSP90 inhibitor include, but are not limited to, viral infection [such as Human Immunodeficiency Virus Type 1 (HIV) infection including acquired immunodeficiency syndrome (AIDS), Influenza infection (Influenza A, influenza B, and Influenza C), Hepatitis B virus (HBV), Hepatitis C virus (HCV), Cytomegalovirus (CMV), Epstein Barr Virus (EBV), Herpes-Simplex Virus (HSV), Varicella-Zoster Virus (VZV), Smallpox virus]; anthrax infection and disease; other bacterial infection [such as Bacterial pneumonia (pneumococcus, Gram negative rod pneumonia, Chlamydia pneumonia, Mycoplasma, Moraxella catarrhalis, Staphylococcus aureus, Streptococcus milleri group), Bacteremia complicated by sepsis or severe sepsis (organ failure) or septic shock, Intraabdominal infection such as diverticulitis, peritonitis, viscus puncture, Clostridium difficile enterocolitis, typhlitis, meningitis, Cardiac infections including bacterial endocarditis], mycobacterial infection [such as Tuberculosis (TB) including multi-drug resistant (MDR) TB and Extremely drug resistant (XDR) TB, Nontuberculous mycobacteria (e.g., M. abscessus, M. Fortuitum, M. Chelonae, M. avium complex (MAC)), M. Kansasii]; fungal infection [such as Candida or aspergillus infection of lung, liver, spleen, sinuses, central nervous system, or sepsis/severe sepsis/septic shock]; graft rejection (e.g., use as suppressing rejection of transplantation of lung, liver, kidney, pancreatic islets, etc.); disease or disorder of joints [such as Rheumatoid arthritis, degenerative joint disease (osteoarthritis), gout, seronegative spondyloarthropathies (i.e., Reiter's syndrome, ankylosing spondylitis, reactive arthritis, psoriatic arthritis); disease or disorder of heart [such as Myocardial infarction (“heart attack”), angina/chest pain, atypical angina, unstable angina, coronary artery disease, atherosclerosis, ischemic cardiomyopathy, congestive heart failure]; disease or disorder of brand or central nervous system [such as cerebrovascular accident (“stroke”), transient ischemic attack (TIA), intracerebral hemorrhage, Parkinson disease, amyotrophic lateral sclerosis, multiple sclerosis, viral or bacterial encephalitis]; disease or disorder of lungs [such as Acute respiratory distress syndrome (ARDS), shock lung, Asthma, chronic obstructive pulmonary disease (COPD), primary pulmonary hypertension, pulmonary embolization]; disease or disorder of liver [such as Shock liver, drug-induced hepatitis, viral hepatitis]; disease or disorder of pancreas [such as acute and chronic pancreatitis, diabetes mellitus type II]; disease or disorder of kidney [Pre-renal azotemia, Acute tubular necrosis (ATN), ischemic nephropathy, glomerulonephritis, Interstitial nephritis, end-stage renal disease (ESRD)]; disease or disorder of gastrointestinal tract [such as ischemic bowel, bowel infarction, inflammatory bowel disease (Crohn disease and ulcerative colitis), Celiac disease (“Sprue”)]; disease or disorder of limbs [such as limb ischemia, limb infarction, thromboangiitis obliterans, Raynaud phenomenon and Raynaud disease, peripheral ulcer disease]; disease or disorder related to coagulation [such as disseminated intravascular coagulopathy (DIC)]; and systemic diseases [such as sarcoidosis, vasculitis, systemic lupus erythematosis (“lupus”)].

In some particular embodiments, methods of the invention include treating an autoimmune disease associated with HSP90. Such autoimmune diseases which are associated with HSP90 include, but are not limited to, skin diseases [e.g., Bullous pemphigoid, Pemphigous vulgaris, Pyoderma gangrenosum, and Hidradenitis suppurativa (acne inversus)], joint diseases (e.g., Rheumatoid arthritis, reactive arthritis, Reiter's syndrome, and Psoriatic arthritis), Vasculitides (e.g., Takayasu's aortitis, Giant cell (temporal) arthritis/Polymyalgia rheumatica, Polyarteritis nodosum (PAN), Thromboangiitis obliterans (Buerger's Disease), Glomerulonephritis, Cryoglobulinemia, Syphilitic vasculitis/aortitis/encephalitis/gummatous destruction, and Wegener's granulomatosis), Gastrointestinal diseases (e.g., Crohn disease and Ulcerative colitis), Systemic diseases (e.g., Systemic lupus erythematosis (“Lupus”), Ankylosing spondylitis, Behçet's disease, Felty's syndrome, Erythema nodosum leprosum, Mixed connective tissue disease, CREST syndrome, Progressive systemic sclerosis (scleroderma), and Sjogren's syndrome), Cardiac disease (e.g., Kawasaki disease), Liver diseases (e.g., Autoimmune hepatitis, Primary biliary cirrhosis, and Sclerosing cholangitis), Kidney diseases (e.g., Goodpasture's syndrome, Wegener's granulomatosis, and IgA nephropathy), Bladder disease (e.g., Interstitial cystitis), Endocrine diseases (e.g., Hashimoto's thyroiditis, Graves' disease, and Addison's disease), Neurological disease (e.g., Guillain-barre syndrome (GBS), Miiler-fisher variant of GBS, and Myasthenia gravis), Hematological diseases (e.g., Idiopathic thrombocytopenic purpura, Thrombotic thrombocytopenic purpura, Autoimmune hemolytic anemia, and Pernicious anemia), and Muscular disease (e.g., polymyositis).

In some embodiments of the invention, methods of the invention include treating a clinical condition that is associated with an excessive level of nitric oxide.

The HSP90 inhibitor can be administered to a patient to achieve a desired physiological effect. The HSP90 inhibitor can be administered in a variety of forms adapted to the chosen route of administration, i.e., orally or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal or respiratory tract inhalation via insufflation and aerosol (also, metered dose inhaler or aerosolized inhaler or powdered inhaler); intraperitoneal; and rectal systemic.

The HSP90 inhibitor can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the HPS90 inhibitor may be incorporated with excipient and used in the form of ingestible tablets, buccal tablets, troches, capsules, elixirs, suspensions, syrups, wafers, and the like. Such compositions and preparation can contain at least 0.1% of HSP90 inhibitor. The percentage of the compositions and preparation can, of course, be varied and can conveniently be between about 1 to about 10% of the weight of the unit. The amount of HSP90 inhibitor in such therapeutically useful compositions is such that a suitable dosage is obtained. Typically, compositions or preparations according to the present invention are prepared such that an oral dosage unit form contains from about 1 to about 1000 mg of HSP90 inhibitor.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the HSP90 inhibitor, sucrose as a sweetening agent, methyl and propylparabens a preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation.

The HSP90 inhibitor can also be administered parenterally. Solutions of the HSP90 inhibitor as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms; however, it should be appreciated that such a preservative need not be present.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacteria and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating an HSP90 inhibitor in the required amount in the appropriate solvent with various other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, typical methods of preparation are vacuum drying and the freeze drying technique which yield a powder of the HSP90 inhibitor plus any additional desired ingredient from previously sterile-filtered solution thereof.

The HSP90 inhibitor can be administered to a subject alone or in combination with pharmaceutically acceptable carriers, as noted above, the proportion of which is determined by the solubility and chemical nature of the compound, chosen route of administration and standard pharmaceutical practice.

Typically, the physician will determine the dosage of the HSP90 inhibitor which will be most suitable for prophylaxis or treatment and it will vary with the form of administration and the particular HSP90 inhibitor chosen, and also, it will vary with the particular patient and disease under treatment. The physician will generally wish to initiate treatment with small dosages by small increments until the optimum effect under the circumstances is reached. The therapeutic dosage can generally be from about 0.1 to about 1000 mg/day, and typically from about 10 to about 100 mg/day, or from about 0.1 to about 50 mg/Kg of body weight per day and typically from about 0.1 to about 20 mg/Kg of body weight per day and can be administered in several different dosage units. Higher dosages, on the order of about 2× to about 4×, may be required for oral administration.

It should also be appreciated that many HSP90 inhibitors are commercially available. Any one of the presently known HSP90 inhibitor can be used in methods of the invention.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Inhibition of cytokine production and inhibition of HIV-1 infection and production by an inhibitor of HSP90, geldanamycin, was examined. The present inventor has observed that geldanamycin potently inhibited Interleukin (IL)-8, IL-6, IL-113, and IL-10 in LPS-stimulated PBMC. In addition, geldanamycin completely inhibited HIV production in both the U1 cell model (HIV production) and the PBMC model (HIV infection and production).

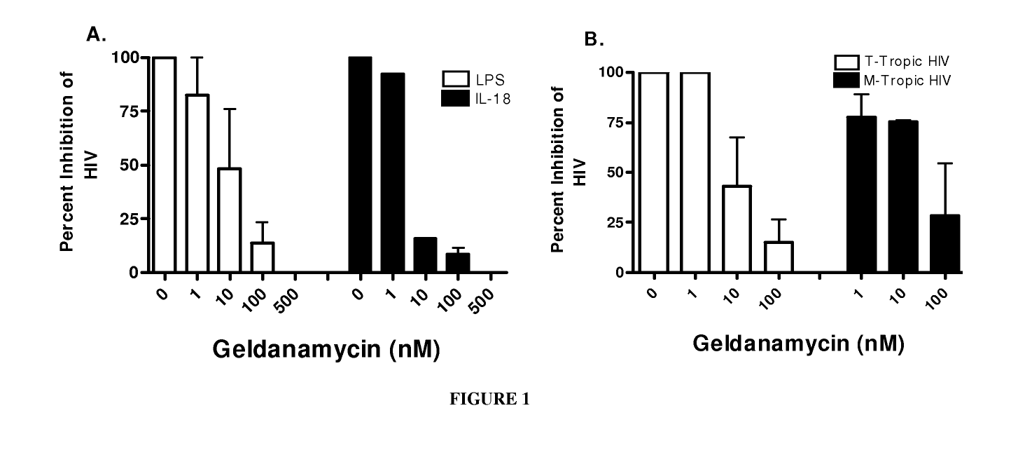

The experiments in both U1 cells and PBMC was extended to determine the dose-response curve for geldanamycin inhibition of HIV. U1 cells were stimulated with either 5 nM IL-18 or 10 μg/ml LPS overnight at 37° C. in the presence or absence of geldanamycin. As shown in FIG. 1A, complete inhibition of LPS- or IL-18-induced HIV production was observed at 500 nM geldanamycin. Geldanamycin was more potent at lower concentrations (10 nM) for IL-18-induced HIV production compared to LPS.

Geldanamycin inhibition of HIV infection of PBMC was also examined. PBMC from five healthy patients were infected for 4 hr with either a T- or M-tropic strain of HIV. Following infection, the PBMC were incubated for 3 days in the presence or absence of geldanamycin. FIG. 1B depicts the results obtained for these infection experiments. While both T- and M-tropic strains were significantly inhibited by 100 nM geldanamycin, the T-tropic HIV strain was also inhibited at 10 nM geldanamycin.

The U1 and PBMC data show that geldanamycin is a potent HIV-1 inhibitor.

### Treatment of Myocardial Infarction (MI or Heart Attack).

For a patient presenting with heart attack as indicated by clinical evaluation, ECG, and cardiac isoenzyme elevation, a patient can be treated with an HSP90 inhibitor given either orally or parenterally (especially intravenously) as soon as the diagnosis of MI is strongly suspected or confirmed. The HSP90 inhibitor medication is given either alone, or, often in conjunction with other medications used to treat acute MI such as tissue plasminogen activator (tPA) or other thrombolytic medication, aspirin, heparin or other anticoagulant, nitrates, statin, angiotensin converting enzyme inhibitor, beta blocker, clopidogrel or other antiplatelet medication, as determined by the attending physician. The HSP90 inhibitor is generally given as a single dose or dosed continuously or intermittently to maintain an appropriate serum concentration (e.g., in the range 1 nM-1,000 micromolar, which is typically determined by clinical experience for optimal effect. HSP90 inhibition can also be used in conjunction with coronary intervention by percutaneous catheterization or with coronary artery grafting surgery.

### Treatment of HIV Infection.

Typically, an oral or intravenous HSP90 inhibitor is used alone, or often in combination with established antiretroviral therapy to suppress replication of the HIV virus that can cause AIDS. This medication is administered in a dose appropriate range (e.g., 1 nM-1,000 micromolar) that is found to be sufficient to suppress replication of the HIV to undetectable levels (e.g., approximately 50 copies/mL, depending on the assay used by the clinician) in the serum of infected patients.

### Treatment of Type 2 Diabetes Mellitus

The anti-inflammatory agent interleukin-1 receptor antagonist has been shown to benefit patients with type 2 diabetes (Larsen et al, New England Journal of Medicine, 2007, vol 365, pages 1517-1526). Since data from the present inventor showed similar anti-inflammatory effects of HSP90 inhibition on inflammatory cytokine production, HSP90 suppression can be used to treat type 2 diabetes. An oral HSP90 suppressant is taken chronically or intermittently in type 2 diabetes patients either alone, or, typically in addition to other glycolytic agents like insulin (injected or inhaled) or oral standard antiglycemic drug(s) like biguanides, sulfonylureas, thiazolidenediones, DPP4 inhibitors, incretin, alpha glucosidase inhibitor, or other such oral drug. An appropriate amount of HSP90 inhibitor is administered (e.g., to produce a serum drug concentration in the range 1 nM-1,000 micromolar).

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for treating a clinical condition associated with biologic activity of heat shock protein 90 (HSP90), said method comprising inhibiting HSP90 by administering to the subject in need of such a treatment a therapeutically effective amount of a composition comprising an HSP90 inhibitor, wherein the clinical condition is selected from the group consisting of systemic disease; bacterial infection; viral disease; fungal infection; a disease or disorder of a joint, cardiovascular system, central nervous system, lung, pancreas, kidney, gastrointestinal tract, or a limb; autoimmune disease; and a combination thereof.

2. The method of claim 1, wherein the bacterial infection comprises infection with mycobacteria, anthrax, bacterial pneumonia, pneumocystis jiroveci pneumonia, or a combination thereof.

3. The method of claim 1, wherein the fungal infection comprises infection of candida, aspergillus, histoplasmosis, or sporothrix schenkei.

4. The method of claim 1, wherein the disease or disorder of a joint comprises rheumatoid arthritis, degenerative joint disease, gout, seroneagative spondyloarthropathies, or a combination thereof.

5. The method of claim 1, wherein the disease or disorder of central nervous system comprises stroke, transient ischemic attack, intracerebral hemorrhage/cerebral vascular accident, Parkinson disease, amyotropic lateral sclerosis, multiple sclerosis, viral or bacterial encephalitis, or a combination thereof.

6. The method of claim 1, wherein the disease or disorder of lung comprises acute respiratory distress syndrome, shock lung, asthma, chronic obstructive pulmonary disease, primary pulmonary hypertension, pulmonary embolization, interstitial fibrosis, or a combination thereof.

7. The method of claim 1, wherein the disease or disorder of liver comprises shock liver, drug-induced hepatitis, viral hepatitis, or a combination thereof.

8. The method of claim 1, wherein the disease or disorder of pancreas comprises acute pancreatitis, chronic pancreatitis, diabetes mellitus type II, or a combination thereof.

9. The method of claim 1, wherein the disease or disorder of kidney comprises pre-renal azotomia, acute tubular necrosis, ischemic nephropaty, glomerulonephritis, interstitial nephritis, acute interstitial nephritis, end-stage renal disease, or a combination thereof.

10. The method of claim 1, wherein the disease or disorder of gastrointestinal tract comprises ischemic bowel, bowel infarction, inflammatory bowel disease, celiac disease, or a combination thereof.

11. The method of claim 10, wherein the inflammatory bowel disease is ulcerative colitis or Crohn disease.

12. The method of claim 1, wherein the disease or disorder of the limb comprises limb ischemia, limb infarction, thromboangiitis obliterans, Raynaud phenomenon, Rheumatoid arthritis, Raynaud disease, peripheral ulcer disease, or a combination thereof.

13. The method of claim 1, wherein the systemic disease comprises sarcoidosis, vasculitis, systemic lupus erythematosis, or a combination thereof.

14. The method of claim 1, wherein the HSP90 inhibitor comprises retaspimycin, tanespimycin, geldanamycin derivative, SNX-2112, SNX-5422, STA-9090, AUY922, or a derivative or a pro-drug thereof, or a combination thereof.

15. The method of claim 1, wherein the HSP90 inhibitor is administered topically, cutaneously, per os (p.o.), intravenously, subcutaneously, intramuscularly, sublingually, or by inhalation.

16. A method for treating a clinical condition associated with excessive nitric oxide, said method comprising inhibiting HSP90 in a subject in need of such a treatment by administering a therapeutically effective amount of a composition comprising an HSP90 inhibitor to reduce the nitric oxide in the subject thereby treating the clinical condition associated with biological activity of nitric oxide.

17. The method of claim 16, wherein the clinical condition associated with nitric oxide activity comprises acquired tubulointerstitial disease, acute respiratory failure, acute respiratory distress syndrome (ARDS), age-associated memory impairment, airway inflammation, amyotrophic lateral sclerosis, asthma, atherosclerosis, autoimmune disease, myocarditis, cerebral ischemia, cerebrovascular disease, chronic liver disease, chronic lung disease, chronic obstructive pulmonary disease, chronic otitis media, congestive heart failure, coronary artery disease, coronary artery ectasia, dysfunctional uterine bleeding, dysmenorrhea, endotoxic shock, end-stage renal disease, falciparum malaria, gastrointestinal pathophysiology, glaucoma, glutamate-induced asthma, glutamate induced Chinese restaurant syndrome, heart failure, heat stress, gastritis, Hirschsprung's disease, hypertension, hypoxemic respiratory failure, inflammatory arthritis, inflammatory bowel disease, inflammatory joint diseases, liver cirrhosis, liver disease, Lyrne neuroborreliosis, migraine, neonatal and pediatric respiratory failure, nephrotoxicity, neurodegenerative diseases, orthopedic disease, osteoarthritis, oxidant stress, Parkinson's disease, pediatric pulmonary disease, pleural inflammation, preeclampsia, primary ciliary dyskinesia, primary pulmonary hypertension, protozoan infections, pulmonary hypertension, retinal disease, septic shock, sickle cell anemia, stroke, systemic lupus erythematosus, traumatic brain injury, or a vascular disease.

18. The method of claim 16, wherein the HSP90 inhibitor comprises retaspimycin, tanespimycin, geldanamycin derivative, SNX-2112, SNX-5422, STA-9090, AUY922, or a derivative or a pro-drug thereof, or a combination thereof.

19. A method for treating a clinical condition associated with HSP90 in a subject, said method comprising administering a therapeutically effective amount of a composition comprising an HSP90 inhibitor to a subject in need of such a treatment, wherein the clinical condition associated with HSP90 is selected from the group consisting of anthrax toxicity, bacterial infection, ischemia-reperfusion injury, and a combination thereof.

20. The method of claim 19, wherein the HSP90 inhibitor comprises retaspimycin, tanespimycin, geldanamycin derivative, SNX-2112, SNX-5422, STA-9090, AUY922, or a derivative or a pro-drug thereof, or a combination thereof.
